(19) United States
(12) Patent Application Publication
(10) Pub. No.: US 2009/0138318 A1
(43) Pub. Date: May 28, 2009

(54) SYSTEMS AND METHODS FOR ADAPTIVE WORKFLOW AND RESOURCE PRIORITIZATION

(75) Inventors: Michael Hawkins, Hoboken, NJ (US); Michael Plavnik, Mahwah, NJ (US); Dmitry Pavlov, New Paltz, NY (US); Alexander Natanzon, Upper Saddle River, NJ (US); Alexander Jurovitsky, Mahwah, NJ (US)

Correspondence Address: HANLEY, FLIGHT & ZIMMERMAN, LLC, 150 S. WACKER DRIVE, SUITE 2100, CHICAGO, IL, 60606, US

(73) Assignee: GENERAL ELECTRIC COMPANY, Schenectady, NY (US)

(21) Appl. No.: 12/275,065

(22) Filed: Nov. 20, 2008

Related U.S. Application Data
(60) Provisional application No. 60/989,378, filed on Nov. 20, 2007.
(60) Provisional application No. 61/015,550, filed on Dec. 20, 2007.
(60) Provisional application No. 60/989,375, filed on Nov. 20, 2007.

(51) Int. Cl. G06F 9/46

(52) U.S. Cl. 705/9; 705/8

(57) ABSTRACT

Certain embodiments of the present invention provide systems and methods for adaptive workflow management in a clinical enterprise. Certain embodiments provide an adaptive clinical workflow management system. The system includes a workflow engine establishing a workflow based on a worklist including a plurality of tasks to be performed. The plurality of tasks are each assigned a priority and relating to patient care. The system also includes a resource monitor monitoring execution of the plurality of tasks in the worklist using one or more clinical resources to determine resource usage. The workflow engine adjusts the priority of tasks in the worklist based on changing system and network conditions to update the worklist and dynamically adjusts resource usage based on the updated worklist.

### RELATED APPLICATIONS

The present application claims the benefit of priority to U.S. Provisional Application No. 60/989,378, filed on Nov. 20, 2007, entitled “Special Methodic for Delivering Media Content”, U.S. Provisional Application No. 61/015,550, filed on Dec. 20, 2007, entitled “Improvement of Diagnostic Reading Efficiency Through Reduction of the Latency Time of Opening New Exam”, and U.S. Provisional Application No. 60/989,375, filed on Nov. 20, 2007, entitled “Special Methodic for Image Handling and Presentation”, each of which is herein incorporated by reference in its entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

Healthcare environments, such as hospitals or clinics, include information systems, such as hospital information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored may include patient medical histories, imaging data, test results, diagnosis information, management information, and/or scheduling information, for example. The information may be centrally stored or divided at a plurality of locations. Healthcare practitioners may desire to access patient information or other information at various points in a healthcare workflow. For example, during and/or after surgery, medical personnel may access patient information, such as images of a patient's anatomy, that are stored in a medical information system. Radiologist and/or other clinicians may review stored images and/or other information, for example.

Using a PACS and/or other workstation, a clinician, such as a radiologist, may perform a variety of activities, such as an image reading, to facilitate a clinical workflow. A reading, such as a radiology or cardiology procedure reading, is a process of a healthcare practitioner, such as a radiologist or a cardiologist, viewing digital images of a patient. The practitioner performs a diagnosis based on a content of the diagnostic images and reports on results electronically (e.g., using dictation or otherwise) or on paper. The practitioner, such as a radiologist or cardiologist, typically uses other tools to perform diagnosis. Some examples of other tools are prior and related prior (historical) exams and their results, laboratory exams (such as blood work), allergies, pathology results, medication, alerts, document images, and other tools. For example, a radiologist or cardiologist typically looks into other systems such as laboratory information, electronic medical records, and healthcare information when reading examination results.

PACS were initially used as an information infrastructure supporting storage, distribution, and diagnostic reading of images acquired in the course of medical examinations. As PACS developed and became capable of accommodating vast volumes of information and its secure access, PACS began to expand into the information-oriented business and professional areas of diagnostic and general healthcare enterprises. For various reasons, including but not limited to a natural tendency of having one information technology (IT) department, one server room, and one data archive/backup for all departments in healthcare enterprise, as well as one desktop workstation used for all business day activities of any healthcare professional, PACS is considered as a platform for growing into a general IT solution for the majority of IT oriented services of healthcare enterprises.

Medical imaging devices now produce diagnostic images in a digital representation. The digital representation typically includes a two dimensional raster of the image equipped with a header including collateral information with respect to the image itself, patient demographics, imaging technology, and other data used for proper presentation and diagnostic interpretation of the image. Often, diagnostic images are grouped in series each series representing images that have some commonality and differ in one or more details. For example, images representing anatomical cross-sections of a human body substantially normal to its vertical axis and differing by their position on that axis from top (head) to bottom (feet) are grouped in so-called axial series. A single medical exam, often referred as a “study” or an “exam” typically includes one or more series of images, such as images exposed before and after injection of contrast material or images with different orientation or differing by any other relevant circumstance(s) of imaging procedure. The digital images are forwarded to specialized archives equipped with proper means for safe storage, search, access, and distribution of the images and collateral information for successful diagnostic interpretation.

Diagnostic physicians that read a study digitally via access to a PACS from a local workstation currently suffer from a significant problem associated with the speed of study opening and making studies available for review where the reading performance of some radiologists requires opening up to 30 MRI studies an hour. Currently, a significant portion of a physician's time is spent just opening the study at the local workstation. When a user is reading one study after another, a switch from a study just read to the next study to be read requires two mouse clicks (one to close the current study and one to open the next study via the physician worklist), introduces delay between those clicks necessary for the refresh of the study list, and an additional delay for loading the next study.

Secondly, current mechanisms for loading a study do not allow for negotiation between instances of a diagnostic viewer that are invoked at the same time and share network bandwidth and processing capability on the workstation trying to simultaneously downloading multiple studies and respond to a user interface reading the study. This causes all studies to load more slowly so that it takes proportionally longer for the first study to become ready for reading. Such an approach is especially detrimental for cases when the first study needs to be downloaded as fast as possible, for example, when reading mammography studies. Bottlenecks develop through inefficient use of available system resources, made worse by a lack of capture of current business and system intelligence.

### BRIEF SUMMARY OF THE INVENTION

Certain embodiments of the present invention provide systems and methods for adaptive workflow management in a clinical enterprise.

Certain embodiments provide a method for adaptive workflow management in a clinical enterprise. The method includes establishing a workflow based on a worklist including a plurality of tasks to be performed. The plurality of tasks are each assigned a priority and relating to patient care. The method also includes monitoring execution of the plurality of tasks in the worklist using one or more clinical resources to determine resource usage. The method further includes adjusting the priority of tasks in the worklist based on changing system and network conditions to update the worklist. The method additionally includes dynamically adjusting resource usage based on the updated worklist.

Certain embodiments provide an adaptive clinical workflow management system. The system includes a workflow engine establishing a workflow based on a worklist including a plurality of tasks to be performed. The plurality of tasks are each assigned a priority and relating to patient care. The system also includes a resource monitor monitoring execution of the plurality of tasks in the worklist using one or more clinical resources to determine resource usage. The workflow engine adjusts the priority of tasks in the worklist based on changing system and network conditions to update the worklist and dynamically adjusts resource usage based on the updated worklist.

Certain embodiments provide a machine readable medium having a set of instructions for execution on a computing machine. The set of instructions includes a workflow engine establishing a workflow based on a worklist including a plurality of tasks to be performed. The plurality of tasks are each assigned a priority and relating to patient care. The set of instructions also includes a resource monitor monitoring execution of the plurality of tasks in the worklist using one or more clinical resources to determine resource usage. The workflow engine adjusts the priority of tasks in the worklist based on changing system and network conditions to update the worklist and dynamically adjusts resource usage based on the updated worklist.

### BRIEF DESCRIPTION OF SEVERAL VIEWS OF THE DRAWINGS

FIG. 1 demonstrates a business and application diagram for PACS information system in accordance with an embodiment of the present invention.

FIG. 2 illustrates an embodiment of an information system delivering application and business content in accordance with an embodiment of the present invention.

FIG. 3 illustrates a block diagram of an example clinical information system that may be used to implement systems and methods described herein.

FIG. 4 shows a block diagram of an example processor system that may be used to implement systems and methods described herein.

FIG. 5 illustrates a flow diagram for a method for transferring images for display at a client workstation in accordance with an embodiment of the present invention.

FIG. 6 illustrates a flow diagram for a method for adaptive workflow management in accordance with certain embodiments of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Certain embodiments relate to system resource and process awareness. Certain embodiments help provide awareness to a user from both a user interface and a client perspective regarding status of a patient and the patient's exam as well as a status of system resources. Thus, the user can review available system resources and can make adjustments regarding pending processes in a workflow. For example, a user may not have printer access to generate a report at a first workstation and may need to log in to another system to generate the report including discharge instructions for a patient and/or feedback for a referring physician. As another example, a certain component or node in an image processing pipeline may be slower than other components or nodes and/or may be experiencing a bottleneck that impacts workflow execution. A user can see, based on system resource and utilization information, when an image is loading slowly and can move on to another task, for example. In certain embodiments, system intelligence can be combined with business intelligence to provide instantaneous vital signs for the organization from whatever desired perspective. Such a combination of system and business intelligence can be used to inform the system and/or user regarding progress of a workflow, status of reporting physicians, how quickly physicians are reacting to information and recommendations, etc. A combination of system and business intelligence can be used to evaluate whether physicians are taking action based on information and recommendations from the system, for example.

As an example related to network connectivity, a client and server can time a period to send and receive data (e.g., compress, send, decompress, and display the data). Given that time, if most of the time is spent compressing and decompressing rather than on the transfer of data, it is likely that the connection between client and server or between two servers may allow a user to attempt transmission in an uncompressed form rather than compressing and decompressing the data for transmission.

In an example implementation, a data transmission can be attempted for a certain number of attempts/period of time (e.g., once), and transmission behavior can then be evaluated to determine whether a time/efficiency savings occurred. If the change was a beneficial change, the system can continue to use that algorithm or approach until conditions change. For example, a clinical information system can send ten megabytes (10 MB) of data via a process of compression, streaming, and decompression. Performance of that transmission can be compared to simply streaming 10 MB of the same data to its destination. Other variables may factor in, such as saturation of the network. For example, transmission may be faster but use more of the available bandwidth, thereby preventing other applications from using that bandwidth (e.g., a particular process or user or set of processes/users may only be allotted 50% or a fixed gigabit of bandwidth within which to transmit multiple streams of data).

A system can have available compression algorithms A and B and can decide between the two based on one or more criteria, for example. If another client workstation is added, the system might have to switch to compression method C based on time, bandwidth, etc. The system can test a change in algorithm in the operational background while the user is working (e.g., can add an extra megabyte of data to the data being transmitted by the user). The system can benchmark, can do a time series until a change is observed, and can then look to see what has changed. Criteria such as processor utilization, network utilization, available compression algorithms, modality, priority (e.g., stat vs. routine), etc., can be analyzed to determine system and network conditions and usability. For example, given a stat case, priorities indicate that a time from request to viewing approaches zero.

Additionally, pervasive adaptive workflow and resource prioritization allows a patient that has been marked as a high priority patient, such as the chief radiologist's daughter, to move to the top of the priority list. Once that patient has been marked as a high priority patient, that's patient's exam and related orders moves to the top of the queue from a system perspective for exam loading, processing, scheduling, labs, etc. Actions of the whole system can be reprioritized based on a change in patient priority, for example.

Thus, certain embodiments provide adaptability and dynamic re-evaluation of system conditions and priorities, enabling the system to react and try different compensating strategies to adapt to changing conditions and priorities.

Certain embodiments relate to reading and interpretation of diagnostic imaging studies, stored in their digital representation and searched, retrieved, and read using a PACS and/or other clinical system. In certain embodiments, images can be stored on a centralized server while reading is performed from one or more remote workstations connected to the server via electronic information links. Remote viewing creates a certain latency between a request for image(s) for diagnostic reading and availability of the images on a local workstation for navigation and reading. Additionally, a single server often provides images for a plurality of workstations that can be connected through electronic links with different bandwidths. Differing bandwidth can create a problem with respect to balanced splitting of the transmitting capacity of the central server between multiple clients. Further, diagnostic images can be stored in one or more advanced compression formats allowing for transmission of a lossy image representation that is continuously improving until finally reaching a lossless, more exact representation. In addition, a number of images produced per standard medical examination continues to grow, reaching 2,500 to 4,000 images per one typical computed tomography (CT) exam compared to 50 images per one exam a decade ago.

Certain embodiments provide “smart” storage, transfer, usability and presentation of diagnostic images to help alleviate certain problems previously found in digital picture archiving and communication systems (PACS) including but not limited to: (1) a load on an information system, (2) a load on a network data transferring system, (3) heavy requirements to image content storage volume; and (4) latency time for image retrieval, image transmission, and image rendering on a diagnostic workstation's display. Additionally, certain embodiments help facilitate improved ergonomic screen layout, image manipulation, and image presentation for a diagnostic physician to provide more effective visual perception and diagnostic reading.

Prior to archiving, digital images are subjected to different types of compression for minimization of required capacity of storage and bandwidth of information links. One compression standard used in medical imaging and other media content information systems is the JPEG2000 standard, which allows decomposition of an image into multiple compression layers, each layer representing a certain scale and resolution of the image. Transmitting a few of the most coarse layers of the image may be used to represent the general image but may compromise the image quality with respect to finer image details. Although coarse image representation is not sufficient for diagnostic visual perception, it still can be suitable for getting a first impression of anatomical scene, navigating through a stack of images to identify a clinically relevant anatomical area, and/or any other interactive preprocessing of the image or whole medical examination. Subsequent transmission and decompression of additional layers enriches the image with fine details and, upon transmission of all layers, reproduce the image in its exact representation.

In certain embodiments, rather than having only one preferred compression scheme (e.g., jpeg, wavelet, layered, etc.), several compression schemes can be provided in a single system, including uncompressed files. Certain embodiments provide a variety of techniques for handling thin slice data. For example, several thin slices and one thick slice can be combined. Certain embodiments utilize similarity of neighboring images to take advantage of common values to compress images. For example, information common to the slices and information regarding differences between the slices are stored so that the image slices can be compressed using an appropriate mechanism and can be delivered to a workstation on request to enable fine grain reading of diagnostic images. Certain embodiments find applicability for both long term and short term storage depending on a combination of price of storage, price of transfer, load on local and remote networks, price of compression (e.g., time), etc.

Certain embodiments provide systems and methods for transfer, improved storage, and improved presentation and perception of diagnostic images and other viewable media in order to help reduce system cost and complexity as well as physician waiting time and to help improve performance and work quality for a physician (and/or other workstation operator) to implement a workflow associated with reading, reviewing, and/or other utilization of the media.

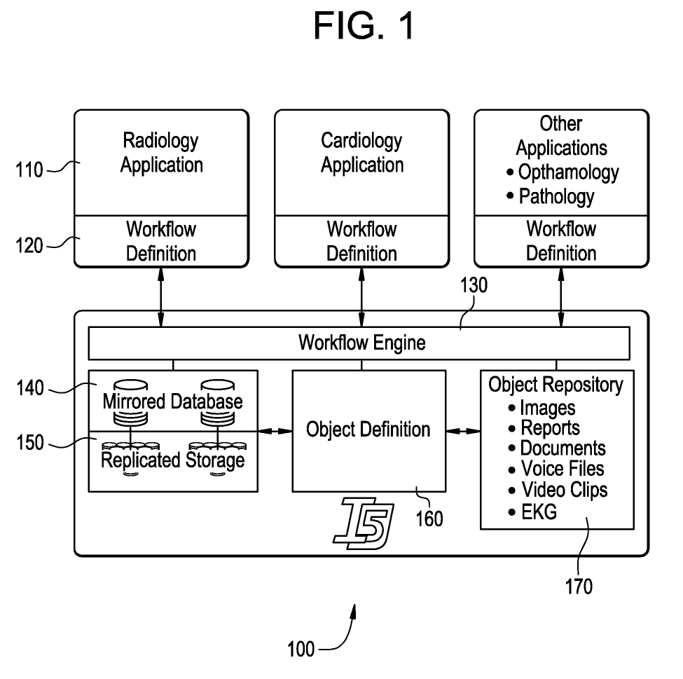

Certain embodiments provide an information system for a healthcare enterprise including a PACS system for radiology and/or other subspecialty system as demonstrated by the business and application diagram in FIG. 1. The system 100 of FIG. 1 includes a clinical application 110, such as a radiology, cardiology, opthalmology, pathology, and/or application. The system 100 also includes a workflow definition 120 for each application 110. The workflow definitions 120 communicate with a workflow engine 130. The workflow engine 130 is in communication with a mirrored database 140, object definitions 60, and an object repository 170. The mirrored database 140 is in communication with a replicated storage 150. The object repository 170 includes data such as images, reports, documents, voice files, video clips, EKG information, etc.

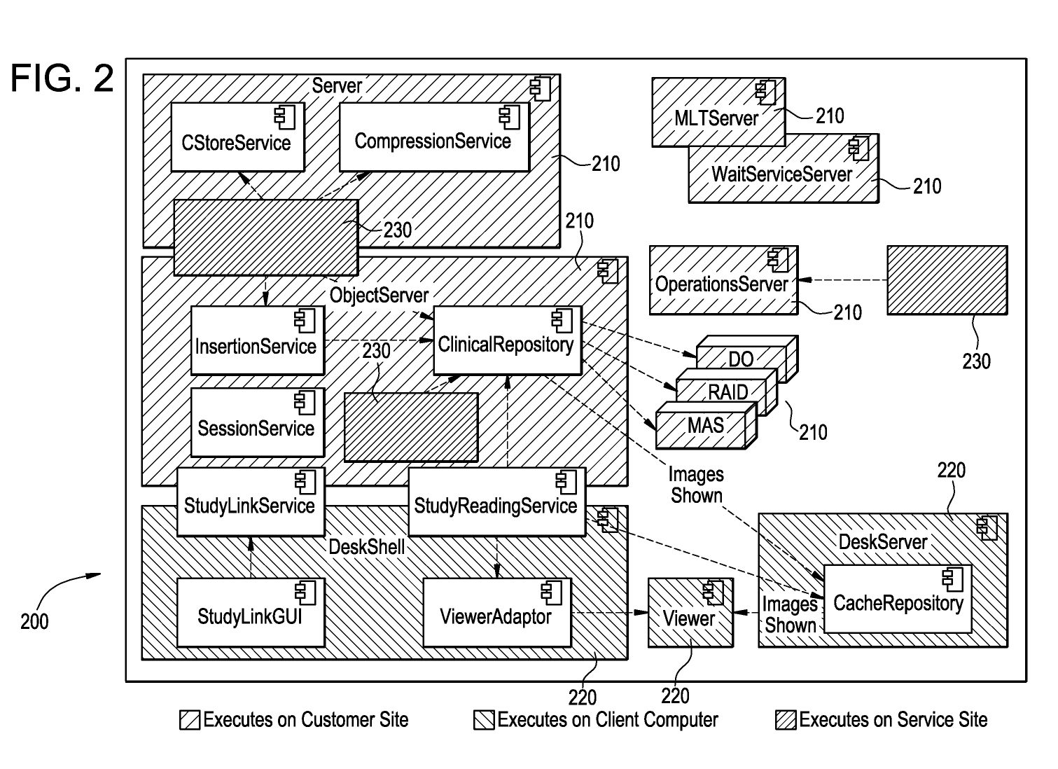

An embodiment of an information system that delivers application and business goals is presented in FIG. 2. The specific arrangement and contents of the assemblies constituting this embodiment bears sufficient novelty and constitute part of certain embodiments of the present invention. The information system 200 of FIG. 2 demonstrates services divided among a service site 230, a customer site 210, and a client computer 220. For example, a Dicom Server, HL7 Server, Web Services Server, Operations Server, database and other storage, an Object Server, and a Clinical Repository execute on a customer site 210. A Desk Shell, a Viewer, and a Desk Server execute on a client computer 220. A Dicom Controller, Compiler, and the like execute on a service site 230. Thus, operational and data workflow may be divided, and only a small display workload is placed on the client computer 220, for example.

Certain embodiments provide an architecture and framework for a variety of clinical applications. The framework can include front-end components including but not limited to a Graphical User Interface (“GUI”) and can be a thin client and/or thick client system to varying degree, which some or all applications and processing running on a client workstation, on a server, and/or running partially on a client workstation and partially on a server, for example.

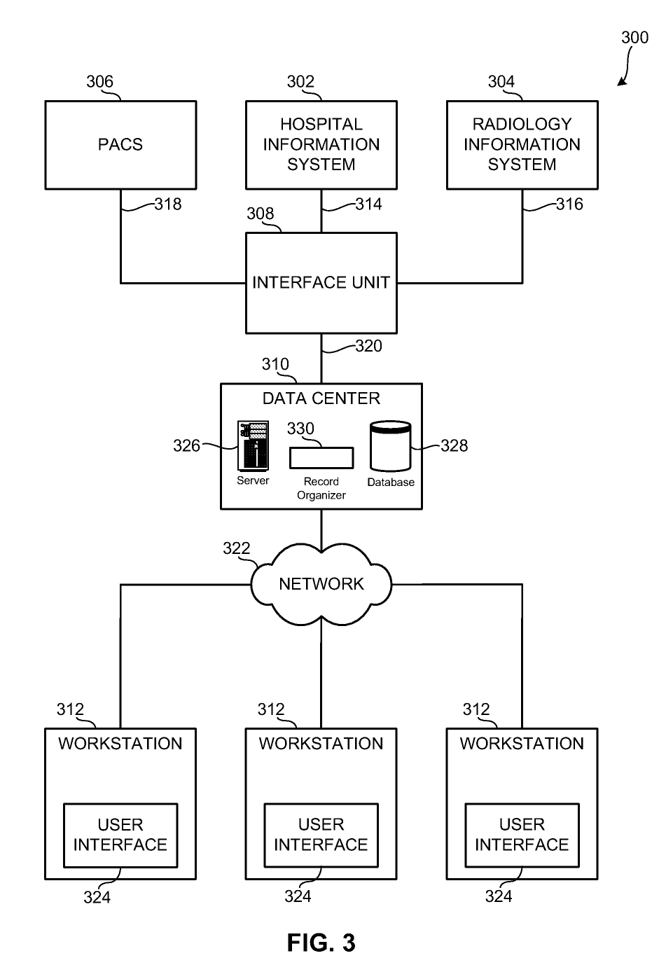

FIG. 3 shows a block diagram of an example clinical information system 300 capable of implementing the example methods and systems described herein. The example clinical information system 300 includes a hospital information system (“HIS”) 302, a radiology information system (“RIS”) 304, a picture archiving and communication system (“PACS”) 306, an interface unit 308, a data center 310, and a plurality of workstations 312. In the illustrated example, the HIS 302, the RIS 304, and the PACS 306 are housed in a healthcare facility and locally archived. However, in other implementations, the HIS 302, the RIS 304, and/or the PACS 306 may be housed one or more other suitable locations. In certain implementations, one or more of the PACS 306, RIS 304, HIS 302, etc., can be implemented remotely via a thin client and/or downloadable software solution. Furthermore, one or more components of the clinical information system 300 may be combined and/or implemented together. For example, the RIS 304 and/or the PACS 306 may be integrated with the HIS 302; the PACS 306 may be integrated with the RIS 304; and/or the three example information systems 302, 304, and/or 306 may be integrated together. In other example implementations, the clinical information system 300 includes a subset of the illustrated information systems 302, 304, and/or 306. For example, the clinical information system 300 may include only one or two of the HIS 302, the RIS 304, and/or the PACS 306. Preferably, information (e.g., scheduling, test results, observations, diagnosis, etc.) is entered into the HIS 302, the RIS 304, and/or the PACS 306 by healthcare practitioners (e.g., radiologists, physicians, and/or technicians) before and/or after patient examination.

The HIS 302 stores medical information such as clinical reports, patient information, and/or administrative information received from, for example, personnel at a hospital, clinic, and/or a physician's office. The RIS 304 stores information such as, for example, radiology reports, messages, warnings, alerts, patient scheduling information, patient demographic data, patient tracking information, and/or physician and patient status monitors. Additionally, the RIS 304 enables exam order entry (e.g., ordering an x-ray of a patient) and image and film tracking (e.g., tracking identities of one or more people that have checked out a film). In some examples, information in the RIS 304 is formatted according to the HL-7 (Health Level Seven) clinical communication protocol.

The PACS 306 stores medical images (e.g., x-rays, scans, three-dimensional renderings, etc.) as, for example, digital images in a database or registry. In some examples, the medical images are stored in the PACS 306 using the Digital Imaging and Communications in Medicine (“DICOM”) format. Images are stored in the PACS 306 by healthcare practitioners (e.g., imaging technicians, physicians, radiologists) after a medical imaging of a patient and/or are automatically transmitted from medical imaging devices to the PACS 306 for storage. In some examples, the PACS 306 may also include a display device and/or viewing workstation to enable a healthcare practitioner to communicate with the PACS 306.

The interface unit 308 includes a hospital information system interface connection 314, a radiology information system interface connection 316, a PACS interface connection 318, and a data center interface connection 320. The interface unit 308 facilities communication among the HIS 302, the RIS 304, the PACS 306, and/or the data center 310. The interface connections 314, 316, 318, and 320 may be implemented by, for example, a Wide Area Network (“WAN”) such as a private network or the Internet. Accordingly, the interface unit 308 includes one or more communication components such as, for example, an Ethernet device, an asynchronous transfer mode (“ATM”) device, an 802.11 device, a DSL modem, a cable modem, a cellular modem, etc. In turn, the data center 310 communicates with the plurality of workstations 312, via a network 322, implemented at a plurality of locations (e.g., a hospital, clinic, doctor's office, other medical office, or terminal, etc.). The network 322 is implemented by, for example, the Internet, an intranet, a private network, a wired or wireless Local Area Network, and/or a wired or wireless Wide Area Network. In some examples, the interface unit 308 also includes a broker (e.g., a Mitra Imaging's PACS Broker) to allow medical information and medical images to be transmitted together and stored together.

In operation, the interface unit 308 receives images, medical reports, administrative information, and/or other clinical information from the information systems 302, 304, 306 via the interface connections 314, 316, 318. If necessary (e.g., when different formats of the received information are incompatible), the interface unit 308 translates or reformats (e.g., into Structured Query Language (“SQL”) or standard text) the medical information, such as medical reports, to be properly stored at the data center 310. Preferably, the reformatted medical information may be transmitted using a transmission protocol to enable different medical information to share common identification elements, such as a patient name or social security number. Next, the interface unit 308 transmits the medical information to the data center 310 via the data center interface connection 320. Finally, medical information is stored in the data center 310 in, for example, the DICOM format, which enables medical images and corresponding medical information to be transmitted and stored together.

The medical information is later viewable and easily retrievable at one or more of the workstations 312 (e.g., by their common identification element, such as a patient name or record number). The workstations 312 may be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. The workstations 312 receive commands and/or other input from a user via, for example, a keyboard, mouse, track ball, microphone, etc. As shown in FIG. 3, the workstations 312 are connected to the network 322 and, thus, can communicate with each other, the data center 310, and/or any other device coupled to the network 322. The workstations 312 are capable of implementing a user interface 324 to enable a healthcare practitioner to interact with the clinical information system 300. For example, in response to a request from a physician, the user interface 324 presents a patient medical history. Additionally, the user interface 324 includes one or more options related to the example methods and apparatus described herein to organize such a medical history using classification and severity parameters.

The example data center 310 of FIG. 3 is an archive to store information such as, for example, images, data, medical reports, and/or, more generally, patient medical records. In addition, the data center 310 may also serve as a central conduit to information located at other sources such as, for example, local archives, hospital information systems/radiology information systems (e.g., the HIS 302 and/or the RIS 304), or medical imaging/storage systems (e.g., the PACS 306 and/or connected imaging modalities). That is, the data center 310 may store links or indicators (e.g., identification numbers, patient names, or record numbers) to information. In the illustrated example, the data center 310 is managed by an application server provider (“ASP”) and is located in a centralized location that may be accessed by a plurality of systems and facilities (e.g., hospitals, clinics, doctor's offices, other medical offices, and/or terminals). In some examples, the data center 310 may be spatially distant from the HIS 302, the RIS 304, and/or the PACS 306 (e.g., at General Electric® headquarters).

The example data center 310 of FIG. 3 includes a server 326, a database 328, and a record organizer 330. The server 326 receives, processes, and conveys information to and from the components of the clinical information system 300. The database 328 stores the medical information described herein and provides access thereto. The example record organizer 330 of FIG. 3 manages patient medical histories, for example. The record organizer 330 can also assist in procedure scheduling, for example.

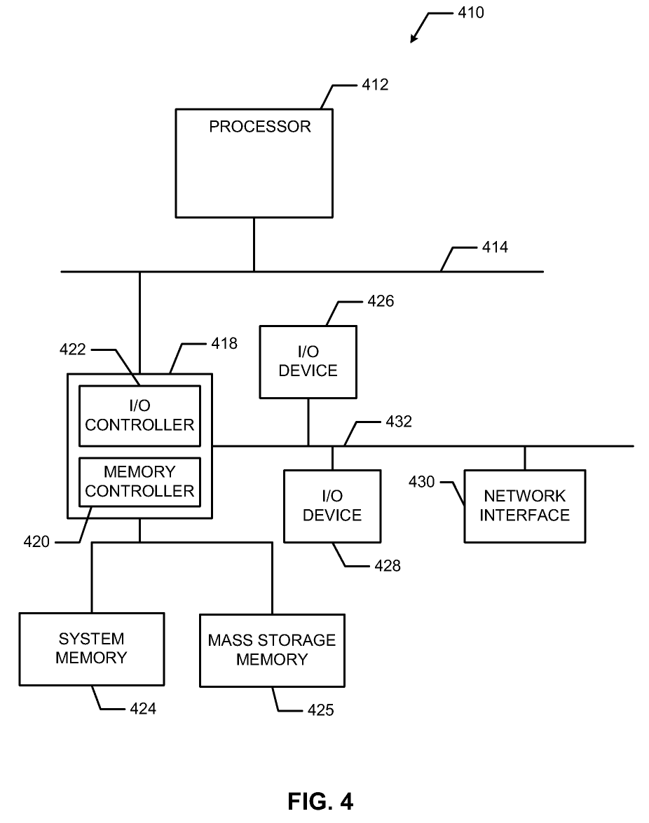

FIG. 4 is a block diagram of an example processor system 410 that may be used to implement systems and methods described herein. As shown in FIG. 4, the processor system 410 includes a processor 412 that is coupled to an interconnection bus 414. The processor 412 may be any suitable processor, processing unit, or microprocessor, for example. Although not shown in FIG. 4, the system 410 may be a multi-processor system and, thus, may include one or more additional processors that are identical or similar to the processor 412 and that are communicatively coupled to the interconnection bus 414.

The processor 412 of FIG. 4 is coupled to a chipset 418, which includes a memory controller 420 and an input/output (“I/O”) controller 422. As is well known, a chipset typically provides I/O and memory management functions as well as a plurality of general purpose and/or special purpose registers, timers, etc. that are accessible or used by one or more processors coupled to the chipset 418. The memory controller 420 performs functions that enable the processor 412 (or processors if there are multiple processors) to access a system memory 424 and a mass storage memory 425.

The system memory 424 may include any desired type of volatile and/or non-volatile memory such as, for example, static random access memory (SRAM), dynamic random access memory (DRAM), flash memory, read-only memory (ROM), etc. The mass storage memory 425 may include any desired type of mass storage device including hard disk drives, optical drives, tape storage devices, etc.

The I/O controller 422 performs functions that enable the processor 412 to communicate with peripheral input/output (“I/O”) devices 426 and 428 and a network interface 430 via an I/O bus 432. The I/O devices 426 and 428 may be any desired type of I/O device such as, for example, a keyboard, a video display or monitor, a mouse, etc. The network interface 430 may be, for example, an Ethernet device, an asynchronous transfer mode (“ATM”) device, an 802.11 device, a DSL modem, a cable modem, a cellular modem, etc. that enables the processor system 410 to communicate with another processor system.

While the memory controller 420 and the I/O controller 422 are depicted in FIG. 4 as separate blocks within the chipset 418, the functions performed by these blocks may be integrated within a single semiconductor circuit or may be implemented using two or more separate integrated circuits.

According to certain embodiments considered as examples in the present application, media files imported from a medical imaging device into a PACS are optionally subjected to a layered incremental compression. Certain media files are grouped in sequences called series, and certain series are grouped into studies, where each study represents a total set of media associated with a single medical exam. Each such study can be optionally attributed to a study type, where each study type is associated with a certain protocol for study interpretation. The protocol can include but is not limited to an order and positions for series display, configuration of a toolbar, annotation and measuring tools, and/or other data required for more efficient presentation of diagnostic images and rendering of a diagnosis. The set of tools and resources is referred to as a “study layout.”

For each study registered in the database, an algorithm (e.g., a unique algorithm) exists for creation of a list of respective series and individual images included in the study and selection of a proper layout for study display. Upon getting a request for study display, the server first generates comprehensive lists of media files to be used for reading the study and a related layout for study display. These lists are transferred to a client workstation and copies are kept on the server. According to the generated list of media files and a chosen layout for their presentation on the client workstation, a plan for transferring and optional processing and/or decompression of the media files is built and coordinated between client and server.

A workflow includes a series of tasks executed to achieve a desired goal. A rule-based workflow system is one whose behavior is codified for particular business data and events. Computer supported cooperative work (“CSCW”) is software designed to allow a group of users on a network to work simultaneously on a project. Groupware can provide services for communicating (e.g., email, text messaging, etc.), group document development, scheduling, and tracking, for example.

In an example digital workflow, a physician order entry (“POE”) is created for a magnetic resonance (“MR”) imaging exam on a specific patient. The workflow includes user authentication, selection of POE order, selection of the patient, and selection of the exam. The workflow further includes entering a clinical indication, entering signs and symptoms, and answering MR safety screening questions to determine priority of exam. The workflow includes reviewing and finalizing of the POE order. In order to define the workflow, a user can define what information must be provided by a referring physician in the POE. For example, how much detail about a specific protocol to be performed needs to be provided. Additionally, will prioritization be graded and transmitted? Will scheduling data be available to the ordering physician. Will the ordering physician be able to select an available exam slot and time at POE order placement? An adaptive workflow system is able to analyze changes to the surrounding operating environment as well as its own operations to select a new response to meet its objectives.

In an adaptive workflow, the system (e.g., a PACS) recognizes that the network between two facilities has been updated to ten gigabyte Ethernet, for example. The system begins sending images to a workstation for review. The system can adapt to send images uncompressed between the two facilities because, with the greater bandwidth, it is now faster to send image data uncompressed than to compress and decompress the information at both sides of the transmission. The system monitors one or more parameters, such as time and bandwidth, and determines whether a change to a compression format should be made over time.

As another example, physicians A and B are both reading a musculoskeletal MR image series. Physician B is also reading a neurological MR image series. An average turnaround time for both physicians is six hours. However, physician B's hours have recently changed. An average turnaround time for the neurological MR now trails an optimal turnaround time by ten hours. A workflow control sets up an interpretation worklist. The control creates a dashboard item to compare turnaround times to an optimal turnaround time. The dashboard notifies the system that the neurological MR has exceeded an optimal turnaround time. The system has access to the same data as the dashboard and is aware of enterprise objectives. The system can adjust exam assignments so that physician B receives the neurological MR series preferentially as well as excess musculoskeletal MRs that cannot be handled by physician A. In addition, depending upon a time of day and/or other environmental factors (e.g., an addition of another neurological MR qualified radiologist), the adaptive system can change the workflow again without involving an administrator. Thus, an adaptive workflow system can combine business intelligence, system intelligence, enterprise objectives, and dashboard/administrative information to adapt and/or otherwise adjust items in a workflow. The adaptive system can change the workflow based on environmental data and other parameters without involving an administrator.

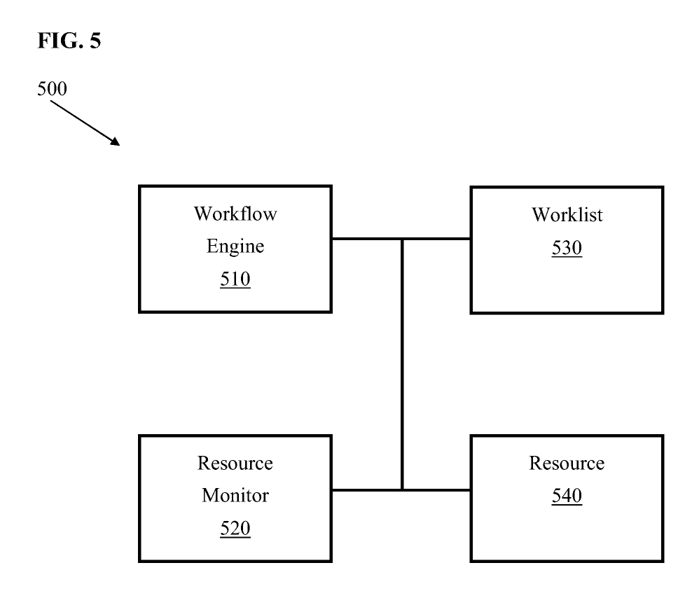

FIG. 5 depicts an adaptive workflow system 500 including a workflow engine 510, a resource monitor 520, a worklist 530, and one or more resources 540. The components of the system 500 can be implemented in software, hardware, and/or firmware, for example. The components of the system 500 can be implemented separately and/or integrated in various forms.

The workflow engine 510 facilitates execution of items on the worklist 530. The worklist 530 is a listing of actions to be done by a clinician to diagnose and/or treat one or more patients. Actions can include image series acquisition, image series review, reporting, laboratory analysis, prescribing, etc. For example, one or more items on the worklist 530 can include identification and transmission of one or more images for display at a client workstation. The workflow engine 510 facilitates data transmission using resource(s) 540 available at a content server, viewing workstation, and communication link between the server and workstation, for example. Based on resource 540 usage, monitored by the resource monitor 520, the workflow engine 510 can dynamically and adaptively adjust execution of actions from the worklist 530 with respect to the resource(s) 540.

Certain embodiments can apply to information and archiving systems used for storage of uniform data or for different types of data and media. Certain embodiments can apply to information systems wherein one of the targets of the information system is delivery of stored data to an application for interactive handling, referred to as “reading,” and implemented though a reading computer application. Reading can be implemented according to a plurality of different scenarios or workflows further referred as “reading workflows.” Each workflow or worklist 530 can involve a different nature of the data set available for reading application. The data set can include data of the same type or of multiple types. Different types of data for each workflow can be of different volume and can be used at different stages of the reading workflow.

Since an information system cannot deliver an entire data set for a reading workflow instantly, certain latency or “idle time” is introduced for an operator implementing the reading workflow. Latency can be caused by, for example, limited bandwidth of communication lines, limited speed of data retrieval from storage media, limited speed for data search, and/or other technological or non-technological limitation.

Certain embodiments reduce latency in delivering images for a reading workflow by providing a loading plan. The plan includes a description of reading workflows implemented and stored. The description can include, for example, a description of data types and data instances for successful running of the reading workflow. The description can also include a technical mechanism used for retrieval and delivery to a reading application of particular data instances for each particular instance of a reading workflow.

The loading plan can also include, for example, a description of what data should be available on a client workstation at any stage of the reading workflow in accordance with its normal implementation or in reaction to interactive actions of the operator. The description can specify data that should be delivered to the workstation for implementation of the next stages of the reading workflow upon most probable interactive interventions of the operator, for example.

Missing data pieces for a next step of the reading workflow are identified, and missing data information is broadcast to all or selected delivery components. For example, delivery hardware and/or software impacted by the missing data are notified about the missing information.

Loading plans governing sequential actions of delivery components can be reconciled in accordance with proceeding to a next stage of the reading workflow and accounting for the missing data. Reconciliations of the loading plans of different delivery elements are coordinated to help reduce or eliminate data traffic congestions through the delivery chain.

In certain embodiments, a variety of factors are considered by the workflow system 500 to adapt a workflow/worklist 530 to changing environmental conditions. For example, device resource issues can include device capabilities (e.g., image display quality), bandwidth (e.g., is it a low bandwidth system), resource type (e.g., a CT scanner type determines preparation time and exam protocols), etc. Personnel issues can include an availability of radiologists for consulting, a desired and/or available skill set, general availability, credentialing and licensing, etc.

Protocol issues can include pre- and/or post-processing, prior exam availability, etc. For example, a 64-slice mulitidetector computed tomography (“MDCT”) protocol requires specific and repetitive 3D post-processing on a dedicated station such that volume images alone should be transmitted to a clinical user. Availability of prior exams may alter the exam protocol and device selected for the exam.

Environmental issues can include connection status, device downtime, etc. For example, if a connection between facilities is lost, the workflow system 500 detects and/or is informed of the lost connection and triggers a priority for local interpretation of image data on-site. Additionally, the system 500 assists in optimizing available resources when a device is down based on one or more factors including urgency, operating room schedule, patient's schedule with referring physician (e.g., seeing the referring physician today rather than tomorrow), underlying condition requiring priority attention, research protocol, etc. Downtime recovery can be partially assisted by creating orders based upon PACS exams and associated information (e.g., examining technician, type of exam, etc.).

Volume issues can include staff, device(s), modality, facility, etc. If one or more of the staff, device(s), modality, and facility are overwhelmed, then tasks can be shifted to other facilities, localities, physicians, interpretation resources, etc. Patient wait times can be monitored by the resource monitor 520 in order to adjust device and personnel work schedules, for example.

Other issues/factors can include awareness of a result and/or of urgent or unexpected findings, patient status, and follow-up of pathology results, for example. In certain embodiments, patient location can be tracked using a device such as a radio frequency identifier (“RFID”). The workflow system 500 can compensate for a patient who has conflicting treatment and/or procedure in real-time or substantially real-time by adjusting a schedule to move patient slots and by saving an empty slot for the patient moving forward. By being aware of patient criticality and an operating room schedule, the workflow engine 510 can prioritize exams based on that schedule. Stat prioritization can be automated to expedite certain operating room studies without manual confirmation, for example.

In operation, for example, the adaptive workflow system 500 can dynamically interpret an operating room schedule. For example, a pre-operative scan is done on a patient. Then, a surgical schedule changes. In the current workflow, the surgical staff calls requesting a priority change to read an exam. The radiology staff changes priority and locates a radiologist to read the exam. In the system 500, the resource monitor 520 notes the change in the surgical schedule. The workflow engine 510 places the exam next on the reading list for the most qualified, available radiologist. The workflow engine 510 notifies the surgical staff that a change has been made in the reading schedule and a new estimated time of arrival has been generated for a clinical report based on the exam reading. The workflow engine 510 is able to access tools and information from the resource monitor 520 to adapt the workflow at least as efficiently as a human administrator, for example.

Thus, an operational efficiency of an enterprise is improved, thereby increasing effectiveness of patient care and quality initiatives, for example. Utilization of available resources such as people, hardware and networks can be maximized rather than adding new components to help with a particular task while other resources lie unused. Additionally, new relationships and tasks can be added to an enterprise timetable rather than a vendor's timetable. New processes can be added that were not considered by a process designer when the system was built or deployed. The adaptive workflow system insulates an enterprise and its users from underlying environmental changes reducing or eliminating a need to adopt disruptive or otherwise inefficient practices.

In certain embodiments, for example, an image transfer and decompression plan prioritizes images such that media images that are currently visible on a client workstation have highest priority for transfer and decompression of fine resolution layers. Thus, images that are currently being reviewed to extract diagnostic features are more quickly transferred and decompressed in their fine resolution representation, so that each currently visible image more quickly reaches diagnostic quality, thus reducing perceived waiting time for a diagnostician.

In certain embodiments, an image transferring and decompression plan prioritizes images such that those images that are currently not visible on a workstation display but have a high probability of becoming visible due to some interactive input of the user (e.g., user scrolling of the image stack) also have sufficiently high priority for transfer and decompression of their fine resolution layers. However, this priority may be lower than a priority for visible image layers, for example. According to one implementation of the transfer and decompression plan, those images reach the client workstation in their diagnostic quality soon after visible images thus reducing the latency time experienced by a diagnostician upon scrolling through the image stack or any other scenario of re-focusing from one diagnostic image to the other. The status changes are communicated to subsystems responsible for image transfer and decompression, triggering changes in implementation of the loading plan by changing priorities of transfer and decompression of individual images in coordination with changing image status.

Thus, certain embodiments provide a PACS configured for importing, storage, access, distribution, and/or interpretation of radiological exams presented as a plurality of digital images and other diagnostic data including but not limited to textual, visual, audio, graphical, and/or other types of data. The PACS can utilize a single data type and/or a combination of different data types with different scenarios for interpretation, management, and/or other functional purposes referred to as workflows, for example. The PACS includes delivery hardware for retrieval of data from physical storage, optional processing of the data, and transferring of the data to a client application. The client application can reside, for example, on the same hardware installation that includes an informational server and physical data storage or on a remote computer connected to the information system through a LAN, WAN, or Internet connection, for example.

The PACS includes one or more delivery engines or dedicated software objects/agents controlling delivery hardware components for delivery of digital data from storage to an end user. Delivery includes but is not limited to retrieval of the data from its original physical storage, optional transmission of the data through a LAN, WAN, Internet, or other connection, and optional compression/decompression or otherwise organized data processing on its way from original physical storage to the end-user client application, for example.

The PACS includes hardware and/or software for creation, storage, and/or modification of data lists including a description of a plurality of data types and a plurality of data objects that should be delivered to a workstation or server for implementation of a workflow. Data lists are retrievable in response to launching a workflow on a workstation or server. Data lists are implemented as explicit lists and/or graphs, recursive lists and/or graphs, on-the fly logical algorithms, and/or other representation, for example.

The PACS establishes one or more loading plans specifying a preferred priority or order for delivery of different objects referred to in a data list to a client/display workstation and/or server in accordance with an implementation of a workflow and/or in accordance with most probable deviations from a workflow implementation resulting from human interactions and/or other factor(s). The PACS can generate a node/engine loading plan for each delivery node/engine based on the general loading plan. The loading plan and/or plans serve as an operational algorithm for the delivery node/engine. The PACS includes software and/or hardware for reconsideration and reconciliation of a general loading plan and node/engine loading plans for relevant delivery nodes/engines in response to proceeding to a next step of the workflow and/or changing of the workflow implementation, for example.

In certain embodiments, the PACS includes a compact or distributed information system designed and used for import, storage, and/or distribution of a uniform data type or data of different natures that includes usage data of a uniform type or a combination of data of different natures within different scenarios (e.g., workflows) of interpretation, management, and/or other functional purpose. The PACS includes delivery hardware from retrieval of data from physical storage, optional processing of the data, and transferring of the data to a client application, provided that the client application can reside either on the same hardware installation that includes an informational server and physical data storage or on remote computer connected to the information system through a LAN, WAN, Internet, and/or other data connection. The PACS includes one or more delivery engines—dedicated software objects/agents controlling delivery hardware component(s) for delivery of digital data from its original storage to an end user. Delivery includes but is not limited to retrieval of the data from its original physical storage, optional transmission of the data through LAN, WAN, Internet, and/or other connection, and optional compression/decompression or otherwise organized data processing as the data is on its way from original physical storage to the end-user application. Delivery agents represented by delivery hardware and delivery engines may be optionally arranged in a chain of delivery nodes such that data used by some delivery node(s) for transfer or processing is provided to the delivery node(s) by another delivery node called a source delivery node, while data processed or transferred by another delivery node is used by a subsequent delivery node called a sink delivery node for further transfer and/or processing. The PACS includes hardware and/or software for creation, storage, and modification of data lists—a comprehensive description of plurality of data types and plurality of data objects that should be delivered to respective workstation or server for successful implementation of the workflow. The data lists are retrieved in response to launching any workflow on any workstation or server implemented either as explicit lists and/or graphs, recursive lists and/or graphs, on the fly logical algorithms, or other means most proper for any preferred embodiment of the invention. The PACS can establish one or more loading plans representing a preferred priority or order for the delivery of different objects referred to in the data list to respective workstation(s) and/or server(s) in accordance with an implementation of a workflow and/or in accordance with one or more most probable deviations from a normal workflow implementation resulting from human interactions and/or any other factors. The PACS generates a node/engine loading plan for each delivery node/engine based on the general loading plan. The loading plan and/or plans serve as an operational algorithm for a delivery node/engine. The PACS can facilitate reconsideration and reconciliation of the general loading plan and/or node/engine loading plans for all relevant delivery nodes/engines in response to a next step in the workflow and/or changing of the workflow implementation. The PACS can coordinate the loading plans for a plurality of delivery nodes/engines along the delivery chain to reduce data congestion along delivery chain. The PACS can synchronize reconciliation of the node loading plans between all delivery nodes serving a workflow. The PACS can adjust a priority, order, and/or speed of data retrieval, transfer, and/or processing by the delivery nodes/engines in response to an updated node loading plan.

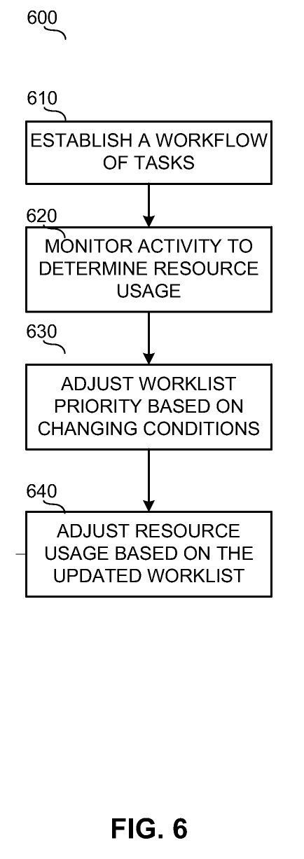

FIG. 6 illustrates a flow diagram for a method 600 for adaptive workflow management in accordance with certain embodiments of the present invention. At 610, a workflow of tasks is established. For example, a worklist can include, for one or more patients, acquisition and delivery of an image series of a patient to a reviewing radiologist, processing and reporting of laboratory tests to an examining physician, etc. Activities in the workflow are executed using resources in one or more clinical systems.

At 620, activity is monitored to determine resource usage. For example, usage and availability of resources in a clinical enterprise are monitored to evaluate which tasks in a worklist are being executed using which resources and whether bottlenecks are occurring or have potential to occur. Certain tasks in a workflow can be assigned a priority such that in the event of a resource conflict or bottleneck, execution of certain tasks take priority over execution of other tasks.

At 630, worklist priority is adjusted based on changing conditions. For example, arrival of a critical patient results in assigning an image series and laboratory test for that patient a priority higher than other non-critical patients in the workflow. Based on resource usage, execution of actions from the worklist can be dynamically and adaptively adjusted with respect to available resources.

At 640, resource usage is adjusted based on the updated worklist. For example, PACS resources to compress, transmit, and decompress images for display are re-tasked to handle an exam order for a critical patient that has been changed to a higher priority in the worklist.

One or more of the steps of the method 600 may be implemented alone or in combination in hardware, firmware, and/or as a set of instructions in software, for example. Certain examples may be provided as a set of instructions residing on a computer-readable medium, such as a memory, hard disk, DVD, or CD, for execution on a general purpose computer or other processing device.

Certain examples may omit one or more of these steps and/or perform the steps in a different order than the order listed. For example, some steps may not be performed in certain examples. As a further example, certain steps may be performed in a different temporal order, including simultaneously, than listed above.

It should be understood by any experienced in the art that the inventive elements, inventive paradigms and inventive methods are represented by certain exemplary embodiments only. However, the actual scope of the invention and its inventive elements extends far beyond selected embodiments and should be considered separately in the context of wide arena of the development, engineering, vending, service and support of the wide variety of information and computerized systems with special accent to sophisticated systems of high load and/or high throughput and/or high performance and/or distributed and/or federated and/or multi-specialty nature.

Certain embodiments contemplate methods, systems and computer program products on any machine-readable media to implement functionality described above. Certain embodiments may be implemented using an existing computer processor, or by a special purpose computer processor incorporated for this or another purpose or by a hardwired and/or firmware system, for example.

One or more of the components of the systems and/or steps of the methods described above may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device. Certain embodiments of the present invention may omit one or more of the method steps and/or perform the steps in a different order than the order listed. For example, some steps may not be performed in certain embodiments of the present invention. As a further example, certain steps may be performed in a different temporal order, including simultaneously, than listed above.

Certain embodiments include computer-readable media for carrying or having computer-executable instructions or data structures stored thereon. Such computer-readable media may be any available media that may be accessed by a general purpose or special purpose computer or other machine with a processor. By way of example, such computer-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions comprise, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of embodiments of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated computer-readable media provide nonvolatile storage of computer-executable instructions, data structures, program modules and other data for the computer.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for adaptive workflow management in a clinical enterprise, said method comprising:
establishing a workflow based on a worklist including a plurality of tasks to be performed, the plurality of tasks each assigned a priority and relating to patient care;
monitoring execution of the plurality of tasks in the worklist using one or more clinical resources to determine resource usage;
adjusting the priority of tasks in the worklist based on changing system and network conditions to update the worklist; and
dynamically adjusting resource usage based on the updated worklist.

2. The method of claim 1, wherein the worklist includes, for one or more patients, at least one of 1) acquisition and delivery of an image series of a patient, and 2) processing and reporting of laboratory tests.

3. The method of claim 1, wherein monitoring comprises monitoring usage and availability of resources to evaluate which tasks in a worklist are being executed using which resources and identify bottlenecks.

4. The method of claim 3, wherein tasks are prioritized in an event of a resource conflict or bottleneck.

5. The method of claim 1, wherein tasks in the worklist for critical patients are prioritized over tasks in the worklist for non-critical patients based on resource usage information.

6. The method of claim 1, wherein monitored resource information comprises one or more of resource capability, communication bandwidth, processor utilization, network utilization, available compression algorithm, modality, priority, and personnel availability.

7. The method of claim 1, wherein monitoring execution comprises benchmarking execution, performing a time series analysis until a change in execution is observed, and reviewing monitor data to determine the change.

8. An adaptive clinical workflow management system, said system comprising:
a workflow engine establishing a workflow based on a worklist including a plurality of tasks to be performed, the plurality of tasks each assigned a priority and relating to patient care; and
a resource monitor monitoring execution of the plurality of tasks in the worklist using one or more clinical resources to determine resource usage;
wherein the workflow engine adjusts the priority of tasks in the worklist based on changing system and network conditions to update the worklist and dynamically adjusts resource usage based on the updated worklist.

9. The system of claim 8, wherein the worklist includes, for one or more patients, at least one of 1) acquisition and delivery of an image series of a patient, and 2) processing and reporting of laboratory tests.

10. The system of claim 8, wherein the resource monitor monitors usage and availability of resources to evaluate which tasks in a worklist are being executed using which resources and identify bottlenecks.

11. The system of claim 10, wherein tasks are prioritized in an event of a resource conflict or bottleneck.

12. The system of claim 8, wherein tasks in the worklist for critical patients are prioritized over tasks in the worklist for non-critical patients based on resource usage information.

13. The system of claim 8, wherein monitored resource information comprises one or more of resource capability, communication bandwidth, processor utilization, network utilization, available compression algorithm, modality, priority, and personnel availability.

14. The system of claim 8, wherein the resource monitor benchmarks execution, performs a time series analysis until a change in execution is observed, and reviews monitor data to determine the change.

15. The system of claim 8, wherein the workflow engine dynamically adjusts resource usage for a subset of a clinical enterprise and propagates the adjustment to the remainder of the clinical enterprise depending upon results in the subset.

16. A machine readable medium having a set of instructions for execution on a computing machine, the set of instructions comprising:
a workflow engine establishing a workflow based on a worklist including a plurality of tasks to be performed, the plurality of tasks each assigned a priority and relating to patient care; and
a resource monitor monitoring execution of the plurality of tasks in the worklist using one or more clinical resources to determine resource usage;
wherein the workflow engine adjusts the priority of tasks in the worklist based on changing system and network conditions to update the worklist and dynamically adjusts resource usage based on the updated worklist.

17. The machine readable medium of claim 16, wherein monitoring comprises monitoring usage and availability of resources to evaluate which tasks in a worklist are being executed using which resources and identify bottlenecks.

18. The machine readable medium of claim 17, wherein tasks are prioritized in an event of a resource conflict or bottleneck.

19. The machine readable medium of claim 17, wherein tasks in the worklist for critical patients are prioritized over tasks in the worklist for non-critical patients based on resource usage information.

20. The machine readable medium of claim 16, wherein monitored resource information comprises one or more of resource capability, communication bandwidth, processor utilization, network utilization, available compression algorithm, modality, priority, and personnel availability.

21. The machine readable medium of claim 16, wherein monitoring execution comprises benchmarking execution, performing a time series analysis until a change in execution is observed, and reviewing monitor data to determine the change.
